(19) United States
(12) Patent Application Publication
(10) Pub. No.: US 2025/0332225 A1
(43) Pub. Date: Oct. 30, 2025

(54) Injectable Neutral pH Collagen-Based Composition For Mesotherapy

(71) Applicant: Shanghai Qisheng Biological Preparation Co., Ltd., Shanghai (CN)

(72) Inventors: Jiaxun ZHU, Shanghai (CN); Li DENG, Shanghai (CN); Hongwei XI, Shanghai (CN); Chen YIN, Shanghai (CN); Wenjie MI, Shanghai (CN); Long ZHANG, Shanghai (CN); Bingbing DONG, Shanghai (CN)

(21) Appl. No.: 19/194,604

(22) Filed: Apr. 30, 2025

Related U.S. Application Data
(60) Provisional application No. 63/640,725, filed on Apr. 30, 2024.

(51) Int. Cl. A61K 38/39; A61K 9/00; A61K 31/167; A61K 31/245

(52) U.S. Cl. CPC A61K 38/39; A61K 9/0019; A61K 31/167; A61K 31/245

(57) ABSTRACT

Provided herein is an injectable neutral pH collagen-based composition for mesotherapy to improve skin appearance, treat skin disease, promote hair growth or reduce cellulite. The composition comprises neutral pH collagen or modified collagen solution; and optionally, biocompatible active ingredient(s) capable of increasing moisturization, preventing skin fine lines formation, enhancing skin brightening, providing anti-aging functions, promoting lipolysis, nourishing follicle cell, promoting blood supply and/or preventing hair loss.

### CROSS-REFERENCES TO RELATED APPLICATIONS

This application claims the benefit of and priority to U.S. Provisional Patent Application 63/640,725, filed on Apr. 30, 2024, which is hereby incorporated by reference in its entirety for all purposes.

### SEQUENCE LISTING

A Sequence Listing conforming to the rules of WIPO Standard ST.26 is hereby incorporated by reference. The Sequence Listing has been filed as an electronic document via PatentCenter in ASCII format encoded as XML.

### FIELD OF INVENTION

The present invention describes injectable neutral pH collagen-based composition for mesotherapy through intra- or sub-cutaneous administration to improve the appearance of skin, treat skin disease, reduce cellulite, and promote hair growth.

### BACKGROUND

Mesotherapy was first reported in 1920s for pain management and anesthesia. Later, the term “mesotherapy” was created by French doctor Dr. Michel Pistor. Word “mesotherapy” comes from the location where treatment is performed, which means structures that were derived from mesoderm rather than itself since the latter ceases to exist beyond the embryonic stage of human development. Mesotherapy has received considerable attention over the past few years. Benefits of mesotherapy include its minimal invasiveness, a mild level of discomfort, and short treatment time. Other advantages claimed for mesotherapy include inexpensive equipment, relatively minimal training needed for operators, prompt and undiluted reach of drugs to the target area, much reduced dosage need of drugs and no necessity for hospitalization. Skin or subcutaneous tissue acts as natural time-release system when drugs are injected by mesotherapy.

Mesotherapy is used to reduce localized pain and to manage some signs and symptoms of chronic venous disease and in some dermatological conditions. Besides these medical applications, it was widely used for cosmetic purposes. Mixture of vitamins, enzymes, hormones, and other substances is administered into the middle layer of skin (dermis) or the fatty tissue just beneath the skin to manage some cases of skin aging, cellulite, and hair loss.

Most compositions used for mesotherapy to achieve skin rejuvenation is based on hyaluronic acid (HA) solution.^{[1]} Several crosslinked HA based mesotherapy products were launched including Belotero ReVive, Restylane Vital, Restylane Vital Light, and Juvéderm® HYDRATE. Another group of products are cocktails of ingredients in non-crosslinked HA solution, such as Cytocare 516, Cytocare 532, Cytocare 640, Cytocare 715, NCTF135® and NCTF135HA®.

The clinical assessment of mesotherapy of crosslinked HA demonstrated improvement of skin hydration, skin elasticity and decreased redness and surface roughness. Ultrasonography showed a dermis thickness and dermis density improvement induced by cocktails of ingredients in non-crosslinked HA solution.^{[2][3]} Hyaluronic acid for skin rejuvenation predominantly effects dermal hydration, associated with reactive oxygen species (ROS) scavenger effect. It is believed that HA based mesotherapy composition restores the mechanical support for fibroblast to optimize its function, and some other ingredients, such as amino acids, coenzymes, minerals and vitamin helps fibroblast perform its biosynthetic activity and have interesting effects on inflammatory process.^{[4]} However, HA based mesotherapy activates metalloproteinase gene expression, leading to an increase in dermal matrix degradation.

Collagen's high biocompatibility makes it a good biomaterial for implantable medical products and scaffolds for in vitro testing systems. Various forms of collagen can be manufactured, such as collagen-based gels, porous sponges, membranes and threads, for surgical and dental purposes or cell culture matrices. These collagen-based compositions have been developed and used for tissue augmentation and drug delivery as described by Dr. Newman and Dr. Weiss.^{[5][6]} Hydrolyzed collagen (collagen peptides) or amino acids (18 essential amino acids, hydroxyproline and hydroxylysine) of collagen was used in mesotherapy solutions as a supplementary component, however, collagen itself was rarely used in mesotherapy because it undergoes fibrillogenesis under neutral pH resulting in poor solubility. Collagen fibrils are difficult for intra- or sub-cutaneous injection via 30 gauge or even smaller needles usually used for mesotherapy, and sometimes results in unnatural-looking white pigmentation when injected in superficial layer of skin. In addition, higher concentrated collagen may leave long lasting bumps after injection.

Though crosslinked collagen-based fillers were developed for soft tissue augmentation, they are not suitable for mesotherapy. Intermolecular cross-links of collagen decrease biodegradation rate by reducing collagen's susceptibility to enzymatic degradation; decrease the capacity of collagen to absorb water; decrease collagen's solubility; and, increase the tensile strength of collagen fibers.

A rapidly polymerizing collagen (RPC) gel, such as that described in U.S. Pat. No. 10,111,981B2, which is non-crosslinked collagen-based filler, was invented for soft tissue augmentation. The RPC gel has high G′ modulus for facial contouring and soft tissue augmentation. However, it is not suitable for mesotherapy injection whether diluted or not. It undergoes fibrillogenesis when diluted with saline or WFI, increasing difficult for injection and cause bump formation.

A micronized equine collagen product in the form of powder placed in vials and sterilized was designed for mesotherapy and reported on PRIME^{[7]}. It shows improvement on skin texture, brightness, hydration, and firmness. However, the micronized and broken collagen fibrils about 3-6 kDa in size lack complete structure and mechanical property of collagen fibrils. The denaturation of triple helix structure of collagen and break of collagen fibrils due to micronization and sterilization cause the exposure of antigens which induce a series of immune response. In tissues, such as skin, tendon, bone, and fascia, collagen exists in tissue in the form of collagen fibers, fibrils, and macroscopic bundles, and is the basis of extracellular matrix (ECM) and the tissue structure, mechanics and function.

Therefore, to achieve satisfactory effect of mesotherapy, there is a need for developing collagen-based compositions with complete triple helix structure and high similarity with ECM for mesotherapy to improve skin appearance, treat skin disease, promote hair growth or reduce cellulite.

### SUMMARY OF THE INVENTION

The collagen-based mesotherapy composition described herein is ideally suited for skin quality improvement. Collagen is the main component of dermis, and both oral and topical collagen can contribute to reducing or delaying skin aging^{[8]}. Collagen with complete triple helix structure benefits fibroblast mitosis and tissue regeneration and new collagen biosynthesis stimulation contributes to skin rejuvenation.

The present application describes injectable neutral pH collagen-based composition for mesotherapy through intra- or sub-cutaneous administration to such as improve the appearance of skin, reduce cellulite, and promote hair growth. Additional active ingredient(s) mixed with or added into the neutral pH collagen matrix of the present invention can further increase moisturization, prevent fine lines formation, enhance skin brightening, provide anti-aging functions, promote lipolysis, nourishing follicle cell, promote blood supply or prevent hair loss.

The present application further describes the formulation and the preparation of injectable neutral pH collagen-based composition for mesotherapy through intra- or sub-cutaneous administration to improve the appearance of skin, treat skin disease, reduce cellulite, and promote hair growth. Collagen solutions are neutral solubilized collagen solution selected from the group consisting of: a) neutralized collagen solution with EDTA and/or disodium citrate and/or polyols. b) redissolved acid collagen solution formed by adding lidocaine/WFI and/or sodium bicarbonate injection solution or sodium citrate injection solution into lyophilized collagen sponge with EDTA and/or disodium citrate and/or polyol and/or active ingredients c) collagen solutions comprising collagens chemically derivatized using one or more agents that react with deprotonated amines and alter the net charge on collagen molecules to adjust the pKa, wherein the collagen maintains its soluble state in solution at physiological pH. Active ingredients mixed with neutral pH collagen solution are intended to increase moisturization, prevent fine lines formation, enhance skin brightening, provide anti-aging functions, promote lipolysis, nourishing follicle cell, promote blood supply or prevent hair loss.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1: SDS-PAGE analysis of neutral pH collagen solution for mesotherapy.

FIG. 2A: exemplary fibrillogenesis curve.

FIG. 2B: fibrillogenesis curve of raw material.

FIG. 2C: fibrillogenesis curve of neutral pH collagen solution for mesotherapy.

FIG. 3: the rheology curves of neutral pH collagen solution for mesotherapy.

FIG. 4: photograph showing a slightly opaque gel formed on the plate of the rheometer.

FIG. 5A: G′ and G″ of polymerized neutral pH collagen solution for mesotherapy.

FIG. 5B: dynamic viscosity of polymerized neutral pH collagen solution for mesotherapy.

FIG. 6A: Dermis and Subcutaneous tissue thickness comparison after 3-time intradermal injection treatment of 0.9% NaCl solution and PDRN collagen solution for mesotherapy.

FIG. 6B: Dermis and Subcutaneous tissue thickness comparison after 3-time intradermal injection treatment of 0.9% NaCl solution and PDRN solution for mesotherapy.

FIG. 6C: Masson's Trichrome Blue stained histopathological image of skin tissue after 3-time intradermal injection treatment of 0.9% NaCl solution.

FIG. 6D: Masson's Trichrome Blue stained histopathological image of skin tissue after 3-time intradermal injection treatment of PDRN collagen solution for mesotherapy.

FIG. 7: Hair growth effect induced by injection of neutral pH collagen solution for mesotherapy.

### DETAILED DESCRIPTION OF THE INVENTION

The following description and examples illustrate embodiments of the invention in detail. It is to be understood that this invention is not limited to the particular embodiments described herein and as such can vary. Those of skill in the art will recognize that there are numerous variations and modifications of this invention, which are encompassed within its scope.

Described herein is a collagen-based biologically compatible composition for mesotherapy which is intended to be injected intra- or subcutaneously. The solubilized neutral pH collagen solution allows intracutaneous and subcutaneous injection using 32˜34 gauge needle or designed automatic or semi-automatic injection system. Additional active ingredient(s) are incorporated with neutral pH collagen solution according to the cosmetic purpose and expected effects including: improving the appearance of skin, treat skin disease, reducing cellulite, and promoting hair growth.

As used herein, the term “soft tissue augmentation” is meant using proper materials and technology to improve, repair or correct facial or body regional volume depletion, and iatrogenic structural soft-tissue malposition. Non-surgical method such as using injectables is often used in where age-related or postsurgical changes manifest as contour or structural visible defects and is available for those who desire volume enhancement for various areas. For example, soft tissue augmentation is conventionally used in wrinkle correction. As used herein, the term “wrinkle”, which is also known as rhytid, is meant a configurational change in skin brought about by repeated muscular contractions associated with genetic and/or environmental factors. Wrinkles can be classified as dynamic or static in nature. Wrinkle is usually measuring deeper than 1 mm and its formation associated with dramatic changes occurred in epidermis (outer layer), the dermis (middle layer), and the subcutaneous tissue (inner layer).

As used herein, the term “mesotherapy” is meant using proper materials and technology to increase moisturization, prevent skin fine lines formation, enhance skin brightening, provide anti-aging functions, promote lipolysis, nourish follicle cell, promote blood supply or prevent hair loss by intra- or sub-cutaneous administration. As compared to materials for soft tissue augmentation, the collagen-based composition for mesotherapy is softer; usually has smaller molecular weight; has lower stiffness and viscosity; provide less or even no shaping force or support force; is administered more superficially. For example, the collagen-based composition for mesotherapy can be used in preventing the formation of fine line rather than correcting wrinkle by augmentation. As used herein, the term “fine line” is meant small, shallow creases on the skin's surface, usually less than 1 mm in depth, which is often caused by repetitive facial expressions, sun damage, dehydration, and the natural aging process.

Active ingredients are water solvent or dispersible in water, isotonic, and non-hypersensitive and don't cause knobs, ulcer or necrosis at infusion site. Since skin is the biggest immune organ full of nerves, the irritation of the collagen-based composition needs be slight or mild and temporary. The collagen-based composition of the present application has low or no irritation and is sterile with very low endotoxin.

By “collagen” is meant all forms of collagen including those which are natural presented, isolated, have been processed or modified. The collagen may be of human or animal origin or may be produced using recombinant techniques. The present invention can use these and other typed of collagen including natural collagen and various collagen derivatives.

Collagen are generally acid soluble and turns into fibrils in neutral pH or under strong ionic strength under 37° C. Fibrils collagen with higher G′ modulus may increase difficulty and pain of injection, and cause unnatural bump under skin. In some embodiments of the present application, to avoid collagen polymerization under neutral condition, chelation agent(s) is added to prevent ions such as calcium, which mediate interactions between collagen molecules and stabilizing the collagen network. In some embodiments, modification of collagen peptides side groups is used to alter the isoelectric point of collagen to improve the solubility of collagen under neutral pH condition. Neutral pH soluble collagen-based compositions are in the form of clear or semi-transparent, injectable, solutions. The neutral pH injectable collagen concentration for intradermal or subdermal injection is at most about 4.8 mg/mL, such as at most 4.8, 4.7, 4.6, 4.5, 4.4, 4.3, 4.2, 4.1, 4.0, 3.5, 3.0, 2.5, 2.0, 1.5, 1.0, 0.9, 0.8, 0.7, 0.6, 0.5 mg/mL, such as about 0.5˜4.8 mg/mL, 0.5˜4.6 mg/mL, 1.0˜4.0 mg/mL, 2.0˜3.8 mg/mL, 2.4˜3.6 mg/mL, or any sub-ranges or points in those ranges. A collagen concentration higher than about 4.8 mg/mL leads to long lasting bump due to collagen fibrillogenesis or collagen deposition induced by fibrillar collagen. A collagen concentration lower than 0.5 mg/mL is not be able to induce suitable collagen fibrillogenesis for mesotherapy.

The active ingredients are mixed with or added to the neutral pH collagen solution during production or preparation before injection include, but are not limited to polyols, natural or derivatized/modified hyaluronic acid, heparosan, glycerin, derivatized chitosan, chondroitin sulfate, dermatan sulfate, nicotinamide for skin moisturization and hydration; botulinum toxin or recombinant botulinum toxin, hexapeptide-3 (Argireline), retinol, ilomastat (ilomostat), 1,2-diacyl-sn-glycero-3-phosphocholine, carnosine, carnitine, glycine, proline, valine for fine lines prevention and neutralization; poly deoxyribonucleic acid (PDRN), polynucleotide (PN), dimethylethanolamine, growth factors, copper peptides for anti-aging effects; crosslinked hyaluronic acid, crosslinked chitosan, hydroxyapatite microspheres, PLLA microspheres, PLGA spheres, PLA-PEG spheres, PCL spheres, PDO spheres for improvement of skin laxity and subcutaneous volume loss; azelaic acid, Vitamin C, glycolic acid, alpha-arbutin, kojic acid, glutathione, lipoic acid, N-acetylcysteine, superoxidase dismutase, methylsulfonylmethane, tretinoin or retinal, dimethicone, nicotinamide, tetrahydrocurminoids for anti-oxidation and skin whitening; brimonidine or tacrolimus for treat skin disease like rosacea; biotin, amino acids, panthenol, caffeine, vitamins (B vitamins, Vitamin C, Vitamin E, Vitamin K), taurine, mineral (calcium chloride, copper sulfate, ferrous sulfate, magnesium sulfate, nickel chloride, sodium metasilicate, zinc sulfate), adenine, linoleic acid, pyruvate, platelet-rich plasma, cell-free fat extract, stem cell-derived exosomes, buflomedil for metabolism and microcirculation promotion, skin and follicle nutrition supply and regeneration enhancement; collagenase, L-carnitine, phosphatidylcholine, sodium deoxycholate, hyaluronidase for cellulite treatment. In addition, active ingredients in nanoparticle form may be included in the collagen-based compositions.

In each case, the active ingredients are added to the collagen solution to a desired concentration. In some embodiments, the active ingredients are basically entrapped in the collagen. Combination of two or more active ingredients need to be compatible in collagen solutions, which is formulated to enhance the cosmetic effect.

In some embodiments, neutral pH soluble collagen-based composition is a neutralized solution or resolubilized lyophilized collagen sponge comprising the acid soluble collagen, EDTA or citrate, wherein the composition is solution at neutralized pH, and soluble collagen polymerizes upon exposure to water or physical fluids. The lyophilized collagen-based composition is resolubilized with water for injection and neutralized with sodium bicarbonate injection solution. Polyol such as mannitol, sorbitol, glycerin is able to maintain the proper osmolality, serve as a lyophilization protecting agent and provide skin hydration properties. Some instable active ingredient including antioxidation ingredient, growth factor, botulinum toxin, poly deoxyribonucleic acid (PDRN), polynucleotide (PN), platelet-rich plasma, cell-free fat extract, stem cell-derived exosomes can be mixed into the collagen solution and lyophilized to avoid the denaturation or inactivation of those instable active ingredient in solution.

In some embodiments, neutral pH soluble collagen-based composition comprises derivatized collagen which is modified by acylation reaction and has been described by DeVore, et. al. in a series of patents (U.S. Pat. Nos. 4,713,446; 4,851,513; 4,969,912; 5,067,961; 5,104,957; 5,201,764; 5,219,895; 5,332,809; 5,354,336; 5,476,515; 5,480,427; 5,631,243; 6,161,544 and 63,188,261). Minerals including calcium chloride, copper sulfate, ferrous sulfate, magnesium sulfate, nickel chloride, sodium metasilicate, zinc sulfate can be mixed and formulated with chemically derivatized collagen solutions to ensure the effects. Collagen solution containing chelation compounds are not proper for minerals formulation.

In certain embodiments, the soluble collagen may be isolated and purified from animal sources including human, axolotl, equine, bovine and porcine tissues or may be a recombinant human collagen produced by mammal cells, microbials, insect cells or plant and plant cells. Collagen solution is not resistant to moist heat sterilization, ethylene oxide sterilization and gamma irradiation. All of them may cause collagen molecule denaturation or molecular chain break. The production of neutral pH soluble collagen-based composition for mesotherapy adopts filtration sterilization and aseptic process to control product sterility. The acid soluble collagen or derivatized collagen solution are sterilized by filtration through 0.45 μm and 0.2 μm filter at a certain low concentration. If desired, collagen concentration can be increased by sterile freeze-drying or diafiltration/ultrafiltration. The other compounds, stabilizer and active ingredients are sterilized and added to the collagen solution for formulation.

### Specific Embodiments

Specific embodiments of the present invention are described again in the following. The present invention in particular also provides for the following items:
1. A method for mesotherapy in a subject in need thereof, comprising intracutaneous or subcutaneous administration a collagen-based composition for mesotherapy through intra cutaneous or subcutaneous injection to a site in need of increasing moisturization, preventing fine lines formation, enhancing skin brightening, providing anti-aging functions, treating skin disease, promoting lipolysis, nourishing hair follicle cell, promoting blood supply or preventing hair loss,
wherein the collagen-based composition for mesotherapy comprises
   (i) 0.5 mg/mL˜4.8 mg/ml neutral pH collagen or modified collagen solution; and optionally
   (ii) 2 mg/mL˜50 mg/ml biocompatible active ingredient(s) capable of increasing moisturization, preventing skin fine lines formation, enhancing skin brightening, providing anti-aging functions, treating skin disease, promoting lipolysis, nourishing hair follicle cell, promoting blood supply or preventing hair loss; and/or
   (iii) local anesthesia drugs for reducing local pain and discomfort during and after treatment.
2. The method of item 1, wherein the composition is administrated intracutaneously or subcutaneously through penetration into a transdermal channel opened by external forces.
3. The method of item 2, wherein the external forces to open transdermal channels are selected from:
   a) using single or array of nano- or micro-needles to puncture skin; and/or
   b) photoelectric treatment including fractional CO₂ laser, picosecond laser, microcurrent treatment, electroporation and radio frequencies; and/or
   c) dermabrasion, chemical peal.
4. The method of item 1, wherein the composition is administrated intracutaneously or subcutaneously through injection by the ways selected from:
   a) traditional single-needle or multi-needle by hands; and/or
   b) auto or semi-auto intradermal injection system; and/or
   c) needle-less intradermal injection system.
5. The method of item 4, wherein the composition is administrated intracutaneously or subcutaneously through 31 gauge, 32 gauge, 33 gauge and/or 34 gauge needle injection; and/or
wherein the average extrusion force of the composition through a 32 gauge needle is ranged from about 2.0 N to about 20.0 N, from about 2.1 N to about 15.0 N, from about 2.2 N to about 14.0 N; and/or
wherein the lag time of the composition is ranged from about 20 minutes to about 90 minutes, from about 20 minutes to about 60 minutes, or from about 20 minutes to about 55 minutes.
6. The method of item 1, wherein the source of collagen for part (i) is selected from allogenetic, mammal hides or marine species or axolotl hides derived matrix; and/or
the collagen is selected from full collagen or atelocollagen, or recombinant collagen or recombinant collagen peptides from microorganism, plants, insect cells or animal cells, or recombinant collagen peptides, or collagen mimic peptides.
7. The method of item 1, wherein the modified collagen is derivatized with acetylation agent(s) that alter the pKa of collagen, and has one or more of the following features:
   (a) soluble at neutral pH;
   (b) does not undergo fibrillogenesis at physiological pH; and/or
   (c) precipitates at acidic pH.
8. The method of item 1, wherein the modified collagen is derivatized with one or more agents selected from the group consisting of glutaric anhydride, succinic anhydride, maleic anhydride, citric acid anhydride, oxalic acid anhydride and ethylenediamine tetraacetic anhydride.
9. The method of item 1, wherein the neutral pH collagen solution comprises a neutralized solution comprising an acid soluble collagen, EDTA, sodium citrate, and wherein the acid soluble collagen comprises collagen selected from the group consisting of Type I collagen, Type III collagen and combinations thereof.
10. The method of item 9, wherein said EDTA is disodium EDTA; and/or
wherein said EDTA is in a concentration between 15 and 50 mM; and/or
wherein said sodium citrate is in a concentration between 50 mM and 150 mM; and/or
wherein said neutral pH collagen solution further comprises a disaccharide, fructose, or combinations thereof; and/or
wherein said rapidly polymerizing collagen gel has an osmolality of 200-400 mOsmol/kg.
11. The method of item 9, wherein the neutral pH collagen solution is a re-dissolved freeze-dried acid soluble collagen solution and neutralized with sodium bicarbonate injection or sodium citrate injection.
12. The method of item 1, wherein the biocompatible active ingredient(s) is one or more selected from the group consisting of:
the biocompatible active ingredient(s) capable of increasing moisturization selected from the group consisting of: polyols, natural or derivatized/modified hyaluronic acid, heparosan, glycerin, derivatized chitosan, chondroitin sulfate, dermatan sulfate, nicotinamide;
the biocompatible active ingredient(s) capable of preventing or neutralizing fine lines formation selected from the group consisting of: botulinum toxin or recombinant botulinum toxin, hexapeptide-3 (Argireline), retinol, ilomastat (ilomostat), 1,2-diacyl-sn-glycero-3-phosphocholine, carnosine, carnitine, glycine, proline, valine;
the biocompatible active ingredient(s) capable of improving skin laxity and subcutaneous volume loss selected from the group consisting of: elastin or tropoelastin, crosslinked hyaluronic acid, crosslinked chitosan, hydroxyapatite microspheres, PLLA microspheres, PLGA spheres, PLA-PEG spheres, PCL spheres, PDO spheres;
the biocompatible active ingredient(s) capable of promoting anti-aging and rejuvenation function selected from the group consisting of: poly deoxyribonucleic acid (PDRN), polynucleotide (PN), dimethylethanolamine, growth factors, copper peptides;
the biocompatible active ingredient(s) capable of helping anti-oxidation and skin whitening selected from the group consisting of: azelaic acid, Vitamin C, glycolic acid, alpha-arbutin, kojic acid, glutathione, lipoic acid, N-acetylcysteine, superoxidase dismutase, methylsulfonylmethane, tretinoin or retinal, dimethicone, nicotinamide, tetrahydrocurminoids;
the biocompatible active ingredient(s) capable of supplying skin and follicle nutrition and enhancing regeneration selected from the group consisting of: decorin, biotin, amino acids, panthenol, caffeine, vitamins (B vitamins, Vitamin C, Vitamin E, Vitamin K), taurine, mineral (calcium chloride, copper sulfate, ferrous sulfate, magnesium sulfate, nickel chloride, sodium metasilicate, zinc sulfate), adenine, linoleic acid, pyruvate, platelet-rich plasma, cell-free fat extract, stem cell-derived exosomes, buflomedil; and
the biocompatible active ingredient(s) capable of treating and remitting cellulite selected from the group consisting of: collagenase, L-carnitine, phosphatidylcholine, sodium deoxycholate, hyaluronidase for cellulite treatment.
13. The method of item 12, wherein the polyols are sugar alcohols selected from glycerin, fructose, xylitol sorbitol, mannitol, allitol, and palatinitol; and/or wherein said polyol is in a concentration between 1.5% and 4% (w/v).
14. The method of item 12, wherein the molecular weight of hyaluronic acid, heparosan and/or chitosan is range from 5 kDa to 100 kDa.
15. The method of item 12, wherein the size of the hydroxyapatite microspheres, PLLA microspheres, PLGA spheres, PLA-PEG spheres, PCL spheres, PDO spheres is ranged from 5 to 150 μm; and/or
wherein the non-resorbable or slowly resorbable spheres are obtained through spray-precipitation technique, emulsion, double emulsion evaporation method, microfluidic reaction, solid-gel process, melt extrusion technique, sintering process or stamp formation; and/or
wherein the non-resorbable or slowly resorbable spheres are sterilized through heat moist sterilization, gamma irradiation or ethylene oxide sterilization.
16. The method of item 1, further comprising local anesthesia drugs such as lidocaine, procaine, preferably in a concentration of from 0.1% to 2% by weight.
17. A collagen-based composition for mesotherapy through intra cutaneous or subcutaneous injection, comprising
   (i) 0.5 mg/mL˜4.8 mg/ml neutral pH collagen or modified collagen solution; and optionally
   (ii) 2 mg/mL˜50 mg/ml biocompatible active ingredient(s) capable of increasing moisturization, preventing skin fine lines formation, enhancing skin brightening, providing anti-aging functions, treating skin disease, promoting lipolysis, nourishing hair follicle cell, promoting blood supply or preventing hair loss; and/or
   (iii) local anesthesia drugs for reducing local pain and discomfort during and after treatment.
18. A method for the preparation of the composition used in the method of any one of items 1˜16, comprising: combining part (i) with part (ii)/(iii), for example by
adding part (ii)/(iii) to part (i) by aseptic mixing;
adding part (iii) to part (i) and part (ii) by using part (iii) injection solution to dissolve the freeze-dried foam of combination of part (i) and part (ii);
adding part (iii) to part (i) and part (ii) by using part (iii) injection solution to dissolve the freeze-dried foam of combination of part (i), using alkali injection solution to dissolve freeze-dried combination of part (ii), mixing two solving liquid together before treatment.

This invention is not limited by the exemplary methods and materials disclosed herein, and any methods and materials similar or equivalent to those described herein can be used in the practice or testing of embodiments of this invention. Numeric ranges are inclusive of the numbers defining the range. The headings provided herein are not limitations of the various aspects or embodiments of this invention which can be read by reference to the specification as a whole.

Throughout the specification, where compositions are described as comprising components or materials, it is contemplated that the compositions can in embodiments also consist essentially of, or consist of, any combination of the recited components or materials, unless described otherwise. Reference to “the disclosure” and “the invention” and the like includes single or multiple aspects taught herein; and so forth. Aspects taught herein are encompassed by the term “invention”.

It is preferred to select and combine preferred embodiments described herein and the specific subject-matter arising from a respective combination of preferred embodiments also belongs to the present disclosure.

### EXAMPLES

### Example 1. Preparation of Neutral pH Modified Collagen Solution for Mesotherapy

Pure type I porcine skin atelocollagen was purchased from Nitta Gelatin, Inc. The collagen was solubilized in water at a concentration of 4.8 mg/mL. The acid solubilized collagen solution was diafiltration and displaced with 0.01M hydrochloric acid at least two times of volume to remove extra impurities and salts. The collagen solution was adjusted to a pH of 9.0 using 10N NaOH under 10° C. and stirred for 15 min at 300 rpm. Pulverized glutaric anhydride powder (Sigma, >95%) was slowly added to the stirring collagen solution at a concentration equal to 10% of the collagen (w/w). The pH of the collagen solution was maintained at pH 9.0 by addition of drops of 10N NaOH. The glutaric anhydride reaction continued for 15 minutes at which point drops of 6N HCl and 1N HCl were added to reduce the pH to approximately 4.5 to precipitate the derivatized collagen. The derivatized collagen was then placed in 50 mL centrifuge tubes and centrifuged at 3,500-5,000 rpm to precipitate the derivatized collagen. The recovered precipitate was then solubilized by adjusting the pH to 7.2 by adding drops of 10N NaOH and 1N NaOH. The derivatized collagen was diluted with water to 4.5 mg/mL. Then the collagen is sterilized by filtration with 0.2 μm PES filters (Pall) and at least 800 mL sterile collagen was collected. 200 mL aqueous solution contained 0.075% NaH₂PO₄, 0.4% Na₂HPO4, 1.5% Lidocaine HCl, 15% mannitol, 1.5% L-carnosine, and 10 mg/mL Vitamin B2, was sterilized by filtration and added to 800 mL the collagen solution and mixed aseptically for over 2 hours under 15° C. and filled into syringes.

### Example 2. Preparation of Neutral pH Collagen Solution for Mesotherapy

Pure type I porcine skin atelocollagen was purchased from Nitta Gelatin, Inc. The collagen was solubilized in water at a concentration of 4.6 mg/mL. The acid solubilized collagen solution was diafiltration and displaced with 0.01M hydrochloric acid at least three times of volume to remove extra impurities and salts. Then the collagen is sterilized by filtration with 0.2 μm PES filters (Pall) and at least 800 mL sterile collagen was collected. 200 ml aqueous solution contained 150 mM EDTA·Na₂, 18% mannitol, 1.1% Na₂CO₃ and 0.9% NaHCO₃ was sterilized by filtration and added to 800 mL collagen solution under aseptic process. Additional 6% Lidocaine HCl solution was sterilized by filtration and add to collagen solution and mixed for over 2 hours under 15° C. and sterile filled into syringes.

### Example 3. SDS-PAGE Analysis of Neutral pH Collagen Solution for Mesotherapy

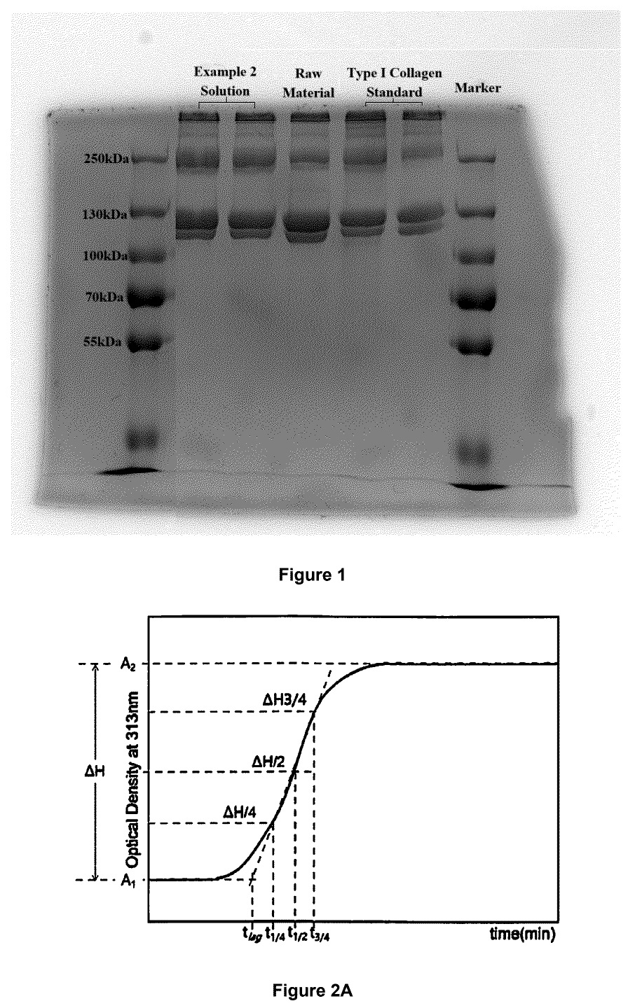

SDS-PAGE analysis was performed on the neutral pH collagen solution for mesotherapy of Example 2. Type I collagen standard (National Institutes for Food and Drug Control of China) and collagen raw material were tested as control. The concentration of stacking gel was 4% (w/v) and the separating gel was 7% (w/v). As shown in FIG. 1, the collagen solution prepared according to the present application shows complete collagen bands (lanes 2 and 3) identical to those of raw material (lane 4) and type I collagen standard (lanes 5 and 6). The result suggests the collagen comprised in the solution of the present application has complete collagen structure.

### Example 4. Fibrillogenesis Analysis of Different Concentration of Neutral pH Collagen Solution

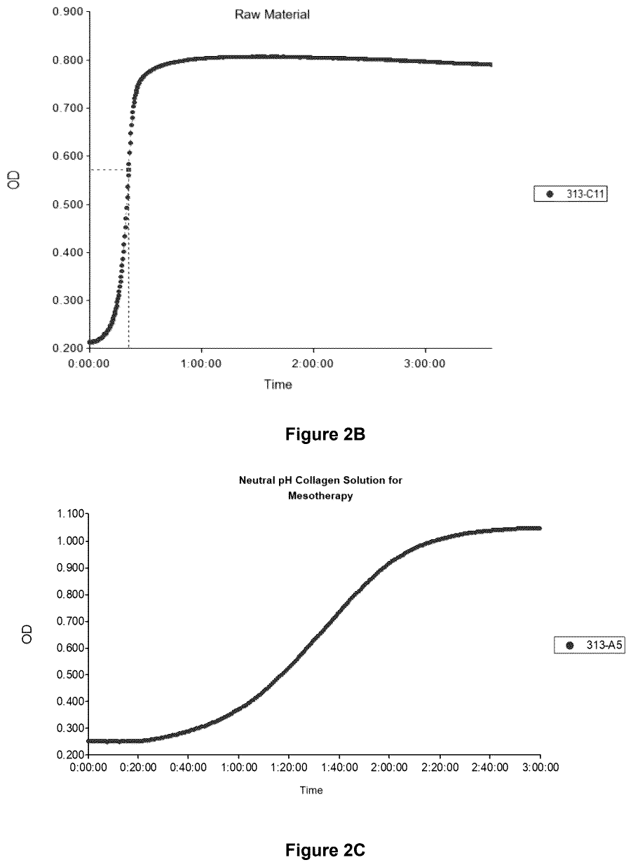

The fibrillogenesis test was performed on collagen raw material and neutral pH collagen solution with different collagen concentration and neutral pH collagen solution with different EDTA and additives concentration for mesotherapy. Table 1 listed all the formulations evaluated in the fibrillogenesis test. Collagen solution was dissolved in 0.01M PBS under 2-8° C. to a one third of the concentration of the original concentration. 200 μL of PBS buffer and the prepared test liquid were added to each hole of the a 37° C. pre-heated 96-well plate. The detection wavelength was set at 313 nm and the temperature was controlled at 37° C. Optical density of the samples was determined (O.D. Value curve over time, take an appropriate interval to record the OD value (20s)) by SYNERGY HTX (BioTek Instruments). The measurement was ended after reaching the platform stability period. FIG. 2A-2C show the OD curve of collagen raw material and neutral pH collagen solution for mesotherapy fibrillogenesis process.

Lag time is calculated as follows (FIG. 2A):

$$Δ H = A 2 - A 1 \qquad ( 1 )$$

where: ΔH—total absorbance change value
A2—Absorbance when the change reaches equilibrium (generally the maximum optical density, or the RSD value of six consecutive points is not greater than 0.05%)
A1—Initial absorbance

$$A 1 / 4 = Δ H \times 1 / 4 + A 1 \qquad ( 2 )$$

$$A 1 / 2 = Δ H \times 1 / 2 + A 1 \qquad ( 3 )$$

$$A 3 / 4 = Δ H \times 3 / 4 + A 1 \qquad ( 4 )$$

By comparing the recorded optical density value with the time data, check the time t corresponding to the optical density value A¼, A½ and A¾ respectively to obtain t¼, t½ and t¾, in minutes. When the optical density value is inconsistent with the corresponding time, the time where the optical density value is closest to it is taken.

Note: t¼, t½, t¾ are the time required for total turbidity change to reach ¼ (ΔH/4), ½ (ΔH/2), and ¾ (ΔH¾), respectively.

According to formula (5), the end time of stasis period t_{lag} in the collagen self-assembly process was calculated. K represented the fibrillogenesis reaction rate.

$${t}_{lag} = \frac{{t}_{1 / 2} \times {t}_{1 / 4}}{{t}_{3 / 4}} \qquad ( 5 )$$

$$K = \frac{Δ H}{2 ( log {t}_{1 / 2} - log {t}_{lag} )} \qquad ( 6 )$$

The lag time of fibrillogenesis of raw material was 16 minutes (FIG. 2B), while the lag time of fibrillogenesis of neutral pH collagen solution for mesotherapy is around 40 minutes (FIG. 2C). The longer fibrillogenesis lag time illustrates that the gelatination of the neutral pH collagen solution for mesotherapy is much more difficult than that of the collagen raw material at the same concentration. This property enables neutral pH collagen solution for mesotherapy to flow and spread after injection to avoid bump forming.

TABLE 1
Example formulations evaluated for fibrillogenesis analysis
and the results of fibrillogenesis analysis

| | Collagen | | EDTA | | Additive | | |
|---|---|---|---|---|---|---|---|
| Exp. | Conc. | | conc. | | Conc. | | |
| # | (mg/mL) | pH | (mM) | Additive | (%) | t_{lag} | K |
| 1 | 3.0 | 2.43 | / | / | / | 14.54 ± 0.51 | 2.35 ± 0.07 |
| 2 | <0.5 | 7.00 | 35 | / | / | Clear, unable to measure | |
| 3 | 3.0 | 7.16 | 35 | / | / | 43.09 ± 1.19 | 2.20 ± 0.09 |
| 4 | 4.5 | 7.11 | 35 | / | 1 | 36.09 ± 2.32 | 2.92 ± 0.08 |
| 5 | 4.8 | 6.96 | 35 | / | / | 33.44 ± 2.62 | 2.98 ± 0.10 |
| 6 | 3.0 | 6.97 | 15 | / | / | 20.96 ± 2.63 | 1.90 ± 0.47 |
| 7 | 3.0 | 7.01 | 25 | | / | 50.98 ± 0.33 | 1.68 ± 0.04 |
| 8 | 3.0 | 7.00 | 25 | Mannitol | 1 | 45.56 ± 3.58 | 2.32 ± 0.12 |
| 9 | 3.0 | 7.03 | 25 | Mannitol | 2 | 41.97 ± 2.66 | 2.18 ± 0.13 |
| 10 | 3.0 | 7.03 | 50 | PDRN | 0.2 | 44.52 ± 1.98 | 2.16 ± 0.08 |
| 11 | 3.0 | 7.02 | 50 | PDRN | 1 | 29.65 ± 2.23 | 2.19 ± 0.12 |

The fibrillogenesis test measured by three repeats and the lag time and the fibrillogenesis reaction rate were calculated and listed in Table 1. Also, the two parameters present the stability of the solution.

Conventional collagen solution (Exp. 1) has acidic pH and a short lag time which are not suitable for mesotherapy. When collagen concentration was lower than 0.5 mg/mL (Exp. 2), the composition is not capable of gelation and is too sick to be used in mesotherapy. Some additives, such as EDTA and mannitol, help to stabilized collagen solution from fibrillogenesis and some additives, such as PDRN, may contribute to fibrillogenesis and can be contained in the neutral pH collagen-based composition.

### Example 5. Thermostability and Rheology of Neutral pH Collagen Solution for Mesotherapy

The thermostability of the neutral pH collagen solution for mesotherapy and raw material acid solution were tested by Discovery DSC25 Auto (TA Instruments). The average denaturation temperature of raw material acid solution was 42.3° C. and the denaturation temperature of neutral pH collagen solution for mesotherapy was 43.8° C. Usually, the concentration of collagen and the polymerization affects the denaturation temperature. In the present test, the raw material acid solution and the neutral pH collagen solution for mesotherapy were at the same concentration. However, the neutral pH collagen solution for mesotherapy remains a clear transparent solution and has a higher denaturation temperature. The result suggests the neutral pH collagen solution for mesotherapy has improved thermostability and maintains its dissolved state in solution at physiological pH.

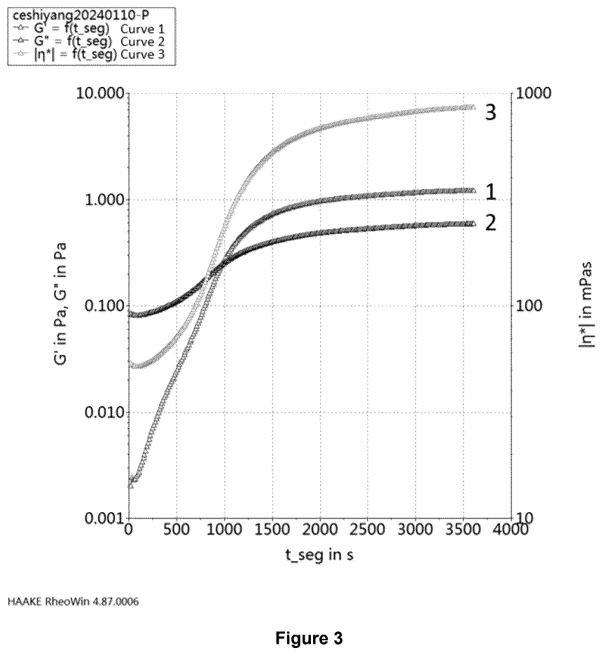

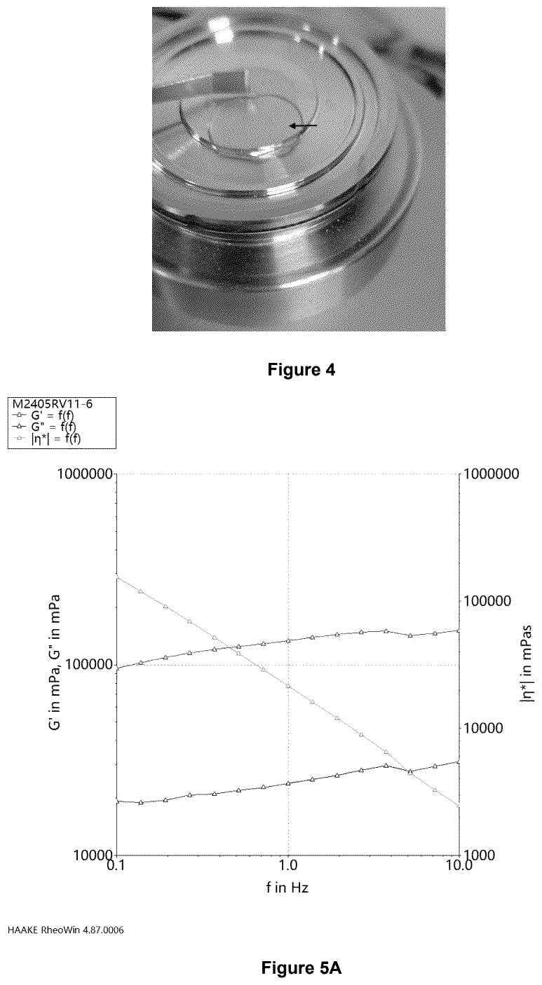

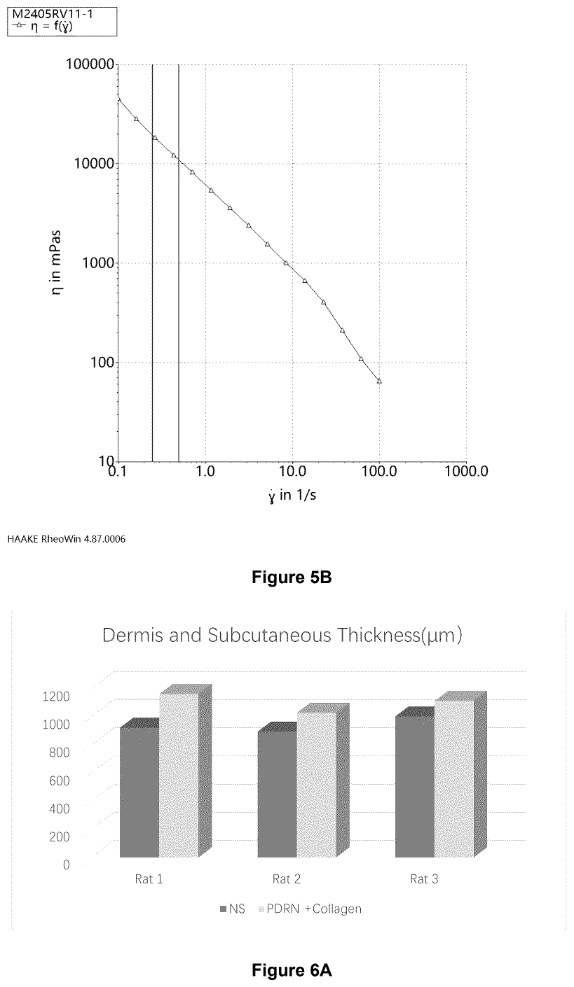

The rheology of neutral pH collagen solution for mesotherapy was measured under the condition of (37±2)° C. and 0.25 Hz with a flat plate (spacing 1 mm) by Haake II rheometer. As shown in FIG. 3, at the beginning, the loss modulus G″ was higher than storage modulus G′, which illustrated that the sample was in a liquid phase. After 1,181 seconds, the storage modulus G′ increased and was higher than the loss modulus G″, which illustrated a phase change from liquid to gel. The crossing point of storage modulus G′ curve and loss modulus G″ curve was the critical point of material gelation. It took around 20 minutes to reach the critical point of material gelation and the G′ of the material was too low to cause unexpected bump under skin after injection. This property allows the solution of the present application spread after injection and integrate with skin tissue later. FIG. 4 is a photo showing a slightly opaque gel formed on the plate of the rheometer at the end of the test. The gel was soft enough to avoid the formation of obvious bumps. (FIGS. 5A and 5B).

The fibrillogenesis of the material and observable gelation macroscopically was a unique property of neutral collagen solution in the present invention. It would avoid material diffusion in vivo and provide continuous improvement after intradermal injection.

### Example 6. Cytotoxicity of Both Neutral pH Modified Collagen Solution and Neutral pH Collagen Solution for Mesotherapy

L929 cell (ATCC) was used for cytotoxicity study of different collagen formulations for mesotherapy. Both collagen solutions produced in Example 1 and Example 2 and their DMEM culture medium containing fetal bovine serum extracts (37° C. incubated for 72±2 hours) were tested according to the method of ISO 10993-5. Solution with lidocaine alone was used as a control.

The results show that Ingredient lidocaine alone has slight cytotoxicity, however, in neutral pH modified collagen solution, lidocaine's cytotoxicity was obscured with the biocompatibility of the solution. Both neutral pH modified collagen solution (Example 1) and neutral pH collagen solution (Example 2) for mesotherapy show very low cytotoxicity which avoid necrosis after injection.

### Example 7. Extrusion Force Measurement of Neutral pH Collagen Solution for Mesotherapy

The samples from Example 2 were filled into 2 mL pre-filled syringe and tested at room temperature. If samples were stored in refrigerator, they should be tested at room temperature after 1 hour of temperature equilibrium. Before the test, supporting core rod and 30G needle (KDL,0.3×13 RWLB), 32G needle (HEALTH BEAUTY, 0.23×8 P TWLB), 34G needle (HEALTH BEAUTY, 0.18×8 P TWLB), 32G 9-needle array (DermaShine, PA-NDL9P32G1), and 34G 4-needle array (MINANK R0.18×1.5 mm) were installed and the air from the front end of the syringe was removed before the syringes were installed on universal test machine (Gotech AI-3000). The extrusion speed was set at 30 mm/min and test distance was full scale, and the maximum, minimum and average extrusion forces were measured and recorded for analysis. 1 mL collagen Filler (Sunmax collagen filler) was dissolved by 8 mL sterile NaCl solution, homogenized by syringe mixing tube for mixing at least 30 times and filled into 2 mL pre-filled syringe for extrusion force test. And a hyaluronic acid solution (WAKE·UP™) with a concentration of 14 mg/mL was filled into 2 mL pre-filled syringe for extrusion force test.

TABLE 2
Maximum, minimum and average extrusion forces of neutral
pH collagen solution for mesotherapy and other samples.

| | | Extrusion | Extrusion | Extrusion |
|---|---|---|---|---|
| | | Force (Max.) | Force (Ave.) | Force (Min.) |
| Example | Gauge | N | N | N |
| neutral pH | 30G Needle | 8.07 ± 0.23 | 7.6 ± 0.22 | 7.18 ± 0.22 |
| collagen | 32G Needle | 12.5 ± 0.62 | 12.3 ± 0.31 | 11.6 ± 0.31 |
| solution for | 34G Needle | 22.9 ± 0.72 | 22.4 ± 0.25 | 21.6 ± 0.19 |
| mesotherapy | 32G 9-Needle Array | 2.72 ± 0.19 | 2.36 ± 0.18 | 2.10 ± 0.20 |
| | 34G 4-Needle Array | 10.0 ± 0.51 | 9.5 ± 0.11 | 9.0 ± 0.11 |
| Collagen | 30G Needle | 20.5 ± 4.53 | 10.2 ± 2.56 | 8.62 ± 3.25 |
| Filler | 32G Needle | 28.5 ± 6.23 | 14.78 ± 3.24 | 13.6 ± 3.89 |
| dissolved to | 34G Needle | 38.6 ± 5.19 | 28.2 ± 4.56 | 25.53 ± 5.12 |
| 3.8 mg/mL | 32G 9-Needle Array | 13.5 ± 3.53 | 8.5 ± 2.15 | 7.9 ± 3.26 |
| by sterile | 34G 4-Needle Array | 24.8 ± 6.53 | 14.92 ± 3.07 | 13.74 ± 2.14 |
| 0.9% NaCl | | | | |
| solution and | | | | |
| homogenized | | | | |
| by syringe | | | | |
| mixing tube | | | | |
| Hyaluronic | 30G Needle | 16.25 ± 0.56 | 15.61 ± 0.90 | 15.10 ± 1.01 |
| Acid solution | 32G Needle | 22.12 ± 0.24 | 21.61 ± 0.33 | 20.98 ± 0.12 |
| for mesotherapy | 34G Needle | 36.25 ± 0.19 | 35.78 ± 0.11 | 35.22 ± 0.31 |
| (14 mg/mL) | 32G 9-Needle Array | 19.25 ± 0.64 | 17.21 ± 0.60 | 16.90 ± 0.76 |
| | 34G 4-Needle Array | 30.44 ± 0.23 | 29.74 ± 0.65 | 28.10 ± 1.01 |

Collagen Filler (Sunmax collagen filler) is a composition containing collagen finished fibrillogenesis, homogenized and resuspended in PBS. It shows greater bias in extrusion force because of its inhomogeneity of collagen fibril formed particles and PBS. As for the hyaluronic acid (HA) solution for mesotherapy, it shows a higher extrusion force because its viscosity.

Instead, the extrusion force of the neutral pH collagen solution for mesotherapy is more stable and lower than those of Collagen Filler and HA solution, which suggests the collagen composition of the present disclosure has excellent solubility and stability and is more suitable for the injection through very tiny needles for mesotherapy.

### Example 8. Rheology of Polymerized Neutral pH Collagen Solution for Mesotherapy

Two grams of sample from Example 2 was added to each well in a 6-well plate and set for polymerization under 37° C. for 4 hours, then 1 mL 0.9% NaCl solution was added to primary polymerized gel in each well to mimic the in vivo physiologic environment and to avoid the evaporation of gel. After incubation for 48 hours, the 0.9% NaCl solution was removed. The G′ and G″ of polymerized gel were measured by HAAKE MARS III using P plate (spacing: 1 mm) under 37° C. with strain of 0.5% and frequency from 0.1 to 10 Hz. The dynamic viscosity of polymerized gel was measured by HAAKE MARS III using P plate (spacing: 1 mm) under 37° C. at shear rate of 10/s with frequency of 0.25 Hz and 0.5 Hz. The G′ and G″ (FIG. 5A) of the polymerized neutral pH collagen solution for mesotherapy are a little bit lower than human dermis, which demonstrates that the solution formed gel would not form obvious bulge after injection. The dynamic viscosity of the polymerized neutral pH collagen solution for mesotherapy suggests that even a bulge formed in dermis, it can be massaged into evenness.

A further rheology analysis on a 5 mg/mL neutral pH collagen solution was conducted in the same way as indicated above. The result shows that the G′ of the neutral pH solution with higher neutral pH collagen concentration reached 1832 Pa under 0.25 Hz, which was almost three times of the G′ of human dermis and will cause a long existing bulge. Combined with the experiments in previous study, neutral pH collagen solutions with a neutral pH collagen concentration ranging from 0.5 to 4.8 mg/ml have much better performance and more effective in mesotherapy.

### Example 9. Injection of Neutral pH Collagen Solution for Mesotherapy in D-Galactose Induced Skin Aging Model of Rat

Total 30 Wistar rats were randomly divided into each experimental group as detailed in Table 3. Animals from each treatment group, except for sham control group, were given subcutaneous injection of D-galactose (D-gal) at a dose of 125 mg/kg·d for 6 weeks to induce skin aging model of rat. At 21 days after D-gal injection, each rat was anesthetized with chloral hydrate (0.25 mL per 100 g body weight) and disinfected within its hip area.

A round tattoo area (d=3 cm) was prefabricated on each side of rat's hip and then treated under general anesthesia once a week for 4 weeks with intradermal microinjection of saline (NS), neutral pH 30 mM EDTA and 2.5% mannitol PBS solution, 0.25 mL neutral pH collagen solution for mesotherapy, 0.5 mL neutral pH collagen solution for mesotherapy, respectively. Both aging negative control and sham control groups received exactly the same surgical procedure but without any treatment under study.

TABLE 3
Details of experimental groups and treatments

| Group # | 1 | 2 | 3 | 4 | 5 |
|---|---|---|---|---|---|
| Group | Sham | Aging Rat | Aging Rat | Aging Rat | Aging Rat |
| | control | Sham | Non-collagen | Low dosage | High dosage |
| Animal | 6 | 6 | 6 | 6 | 6 |
| number | | | | | |
| week | / | D-gal | D-gal | D-gal | D-gal |
| 1-6 | | 125 mg/kg · d | 125 mg/kg · d | 125 mg/kg · d | 125 mg/kg · d |
| week | / | / | / | / | / |
| 7-9 | | | | | |
| week | 0.9% NaCl | 0.9% NaCl | neutral pH | neutral pH | neutral pH |
| 10-13 | | | 30 mM EDTA | collagen | collagen |
| | | | and 2.5% | solution for | solution for |
| | | | mannitol PBS | mesotherapy | mesotherapy |
| | | | solution | | |
| | 0.5 mL/ | 0.5 mL/ | 0.5 mL/ | 0.25 mL/ | 0.5 mL/ |
| | 100 g · week | 100 g · week | 100 g · week | 100 g · week | 100 g · week |
| week | / | / | / | / | / |
| 14-17 | | | | | |

| | |
|---|---|
| week 18 | Euthanized |

28 days after treatment, all animals were euthanized and skin samples were compared by histology, and quantitative real-time PCR testing.

Aging skins show decreased thickness for both epidermis and dermis when compared to sham control rats. With aging, the epidermis reduces its numbers of cell layers, and the dermal collagen fibers turn to be sparse, slender, or broken. When compared to aging control, however, these histologic changes were greatly ameliorated in neutral pH collagen solution for mesotherapy groups (Table 4). When treated with neutral pH collagen solution for mesotherapy, both epidermal and dermal thickness and density of collagen fiber were significantly elevated over those of aging control group. Moreover, the dermal thickness and density of collagen fiber increasing showed a dose-dependent effect with the concentration of neutral pH collagen solution for mesotherapy. Collagen type I expression was greatly upregulated in response to both 0.25 mL neutral pH collagen solution for mesotherapy and 0.5 mL neutral pH collagen solution for mesotherapy which showed no significant difference to that of sham control. However, any of the other aging groups showed much less expression of collagen type I when compared to sham control rats.

TABLE 4
Histology and collagen content of skin in different groups

| Group # | 1 | 2 | 3 | 4 | 5 |
|---|---|---|---|---|---|
| Epidermal Thickness (μm) | 80.4 ± 0.4 | 48.5 ± 0.2 | 52.1 ± 0.5 | 61.3 ± 0.5 | 63.5 ± 0.7 |
| Dermal Thickness (μm) | 663 ± 24 | 412 ± 13 | 435 ± 24 | 523 ± 18 | 521 ± 32 |
| Collagen fiber Density (mm²) | 0.43 ± 0.12 | 0.15 ± 0.05 | 0.21 ± 0.08 | 0.26 ± 0.10 | 0.32 ± 0.09 |
| Water Content (%) | 0.65 ± 0.02 | 0.63 ± 0.04 | 0.63 ± 0.05 | 0.64 ± 0.05 | 0.65 ± 0.04 |
| Hydroxyproline Content | 1.62 ± 0.13 | 1.24 ± 0.07 | 1.26 ± 0.10 | 1.41 ± 0.05 | 1.48 ± 0.06 |
| (μg/mg pro) | | | | | |

When treated with 0.25 mL neutral pH collagen solution for mesotherapy, 0.5 mL neutral pH collagen solution for mesotherapy and neutral pH 30 mM EDTA and 2.5% mannitol PBS solution, transcript levels for collagen type III were considerably elevated over aging control group, but significantly less than sham control. As a potential key regulator in skin aging, MMP-1 messages increased in response to either 0.25 mL or 0.5 mL neutral pH collagen solution for mesotherapy, which showed a statistically significant difference from that of aging control, but not sham control rats. In contrast, transcript levels for TIMP-1 in all aging groups were significantly downregulated to a much lower level of that of sham control.

TABLE 5
Design of RT-PCR primers and data collection

| Primer pairs for target gene | | | | | | | | |
|---|---|---|---|---|---|---|---|---|
| for RT-PCR assay | | | | | | | | |
| | FW | RV | Probe | Expression ΔΔCt | | | | |
| | (SEQ | (SEQ | length | Group | Group | Group | Group | Group |
| Target Gene | ID # | ID #) | (bp) | 1 | 2 | 3 | 4 | 5 |
| Col α1 I | 1 | 2 | 203 | 5.0 ± 0.6 | 1.0 ± 0.1 | 1.5 ± 0.3 | 3.1 ± 0.2 | 3.5 ± 0.3 |
| NM_053304.1 | | | | | | | | |
| Col α1 III | 3 | 4 | 132 | 6.8 ± 0.5 | 1.0 ± 0.1 | 1.8 ± 0.1 | 2.8 ± 0.2 | 3.1 ± 0.1 |
| NM_032085.1 | | | | | | | | |
| MMP-1α | 5 | 6 | 227 | 2.4 ± 0.5 | 1.0 ± 0.1 | 1.2 ± 0.2 | 2.0 ± 0.2 | 2.3 ± 0.2 |
| NM_001134430.1 | | | | | | | | |
| TIMP-1 | 7 | 8 | 191 | 3.6 ± 0.5 | 1.0 ± 0.1 | 1.3 ± 0.2 | 1.2 ± 0.2 | 1.5 ± 0.2 |
| NM_053819.1 | | | | | | | | |
| β-Action | 9 | 10 | 150 | 2.5 ± 0.3 | 1.8 ± 0.3 | 2.1 ± 0.1 | 1.9 ± 0.3 | 2.0 ± 0.2 |
| NM_031144.3 | | | | | | | | |

The results suggested that neutral pH collagen solution directly increases the epidermis and dermis density and thickness, up-regulates ECM synthesis and reverses the skin aging state to a health and youthful state.

### Example 10. Lyophilized PDRN Collagen Solution for Mesotherapy

Pure type I porcine skin atelocollagen was purchased from Nitta Gelatin, Inc. The collagen was solubilized in water at a concentration of 4.8 mg/mL. The acid solubilized collagen solution was diafiltration and displaced with 0.01M hydrochloric acid at least two times of volume to remove extra impurities and salts. Then the collagen is sterilized by filtration with 0.2 μm PES filters (Pall) and at least 800 mL sterile collagen was collected. 200 ml solution containing 150 mM EDTA·Na₂, 18% mannitol, 0.5% Na₂CO₃ and 1.8% PolyDeoxyRiboNucleotide (PDRN, HTL Biotechnology PDRN, MW>500 kD) was sterilized by filtration and added to collagen solution under aseptic process and mixed for over 2 hours under 15° C. and sterile filled into penicillin bottles at a volume of 5 mL. The solution was lyophilized under a process with a precooling under −45° C. (0.5° C./min cooling down and maintained for 4 hours) with annealing at −20° C. (1° C./min heating down and maintained for 2 hours), cooling under (0.5° C./min cooling down and maintained for 2 hours) and primary freeze-drying at 0° C. for no less than 12 hours and secondary freeze-drying at 5° C. for no less than 12 hours.

The freeze-dried PDRN collagen was resolubilized with 1 mL 2% lidocaine and 2 mL 5% sodium bicarbonate injection solution before treatment.

### Example 11. Injection of Lyophilized PDRN Collagen Solution for Mesotherapy in Normal Rats

Total 6 Sprague Dawley rats were randomly divided into each experimental group. For the first group (n=3), a round area (d=3 cm) was prefabricated on each side of rat's back and then treated under general anesthesia once a week for 3 weeks with intradermal microinjection of saline (NS) on the left back and neutral pH PDRN solution (same concentration of 0.6%) on the right of the back. For the second group, a round area (d=3 cm) was prefabricated on each side of rat's back and then treated under general anesthesia once a week for 3 weeks with intradermal microinjection of saline (NS) on the left back and neutral pH PDRN collagen solution for mesotherapy on the right of the back.

21 days after treatment, all animals were euthanized and skin samples around injection sites were compared by histology.

Based on the observation of pathological section of the skin samples, there were few collagen samples detected in the tissue. The histological of skin sample shows around 10% increase of dermis and subcutaneous tissue thickness after treatment by PDRN collagen solution for mesotherapy comparing to the skin treated by saline and neutral pH PDRN solution.

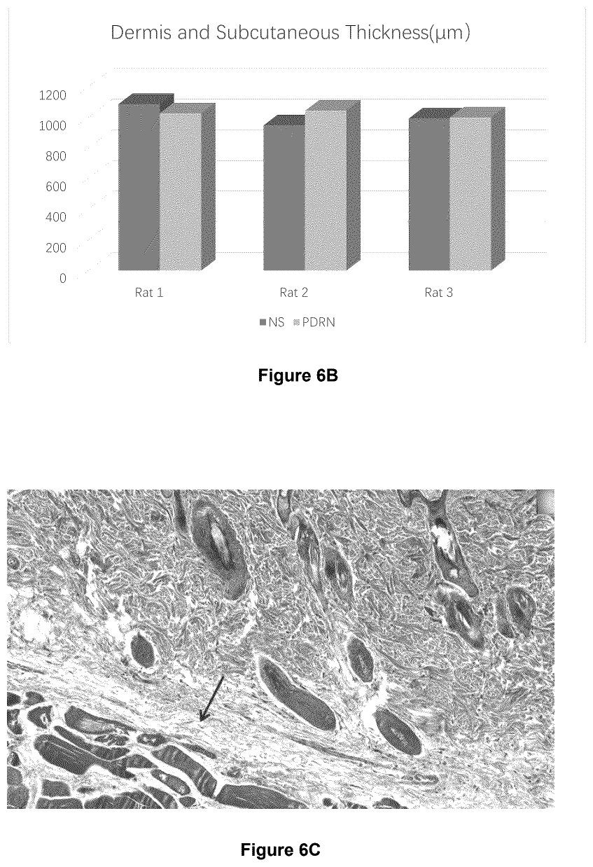

The results suggest that PDRN collagen solution has a better performance for improvement of skin laxity and subcutaneous volume loss than PDRN solution only (FIGS. 6A and 6B). In addition, the Masson Trichrome Blue staining shows increasing of collagen fibrils density between the deep dermis and above subcutaneous muscle (FIGS. 6C and 6D).

### Example 12. Hair Growth Enhancement by Injection of Neutral pH Collagen Solution for Mesotherapy

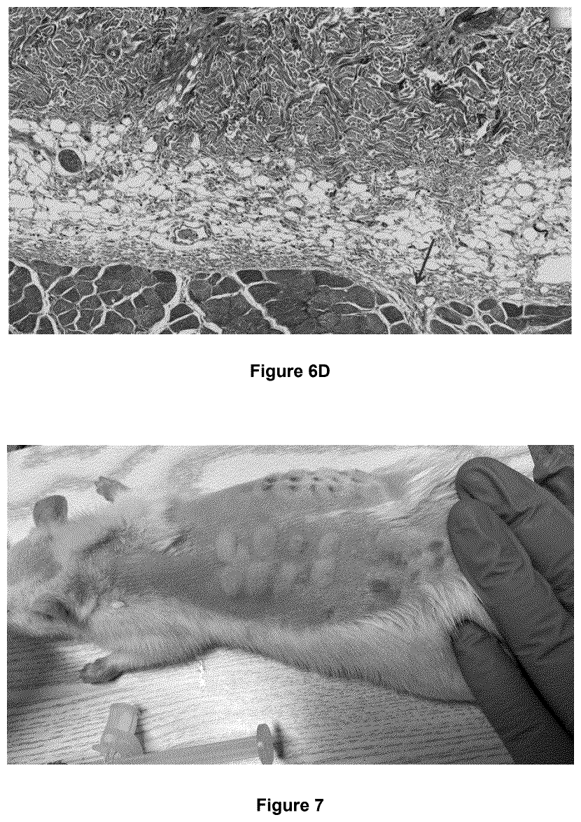

Sprague Dawley rat was prefabricated on each side of rat's back and then treated under general anesthesia once a week for 2 weeks with intradermal microinjection of saline (NS) on the left back and neutral pH collagen solution for mesotherapy. Obvious hair growth was observed on the right back with neutral pH collagen solution treatment comparing to the left side (FIG. 7). The injection of neutral pH collagen solution provides a better ECM for hair follicles and promotes hair growth. The results suggest neutral pH collagen solution can be used as hair follicles and hair growth stimulator to enhance hair growth.

Although the present invention has been described with reference to exemplary embodiments, one skilled in the art can easily ascertain its essential characteristics and without departing from the spirit and scope thereof, can make various changes and modifications of the invention to adapt it to various usages and conditions. Those skilled in the art will recognize or be able to ascertain using no more than routine experimentation, many equivalents to the specific embodiments of the invention herein. Such equivalents are intended to be encompassed in the scope of the present invention.

All references, including patents, publications, and patent applications, mentioned in this specification are herein incorporated by reference in the same extent as if each independent publication, patent or patent application was specifically and individually indicated to be incorporated by reference.

### NON-PATENT REFERENCES

[1] Fatima A, Faresi & Hassan, Galadari (2011) Mesotherapy: myth and reality, Expert Review of Dermatology, 6:2, 157-162.
[2] Grand-Vincent, A., Boisnic, S., Salomon, C., Prinderre, P. and Piccerelle, P. (2017) Clinical Assessment of a Mesotherapy Formulation for Skin Rejuvenation in Healthy Volunteers. Journal of Cosmetics, Dermatological Sciences and Applications, 7, 291-305.
[3] Hertz-Kleptow D, Hanschmann A, Hofmann M, Reuther T, Kerscher M. Facial skin revitalization with CPM®-HA20G: an effective and safe early intervention treatment. Clin Cosmet Investig Dermatol. 2019 Aug. 13; 12:563-572.
[4] Prikhnenko S. Polycomponent mesotherapy formulations for the treatment of skin aging and improvement of skin quality. Clin Cosmet Investig Dermatol. 2015 Apr. 7; 8:151-7.
[5] James Newman (2009) Review of soft tissue augmentation in the face, Clinical, Cosmetic and Investigational Dermatology, 2:, 141-150.
[6] Friess W. Collagen—biomaterial for drug delivery. Eur J Pharm Biopharm. 1998 March; 45(2):113-36.
[7] Skin rejuvenation with equine collagen type 1|PRIME Journal (prime-journal.com) https://www.prime-journal.com/skin-rejuvenation-with-equine-collagen-type-1/.
[8] Al-Atif H. Collagen Supplements for Aging and Wrinkles: A Paradigm Shift in the Fields of Dermatology and Cosmetics. Dermatol Pract Concept. 2022 Jan. 1; 12 (1):e2022018.

### PATENT REFERENCES

U.S. Pat. No. 10,111,981B2; U.S. Pat. Nos. 4,713,446; 4,851,513; 4,969,912; 5,067,961; 5,104,957; 5,201,764; 5,219,895; 5,332,809; 5,354,336; 5,476,515; 5,480,427; 5,631,243; 6,161,544; and 63/188,261

Sequence information:

SEQ ID NO: 1: atgtctggtttggagagagca
SEQ ID NO: 2: gaggagcaggacttcttgag
SEQ ID NO: 3: gcctcccagaacattacatacc
SEQ ID NO: 4: tttgctatttccttcagccttg
SEQ ID NO: 5: ctcccttggactcactcattcta
SEQ ID NO: 6: agaacatcacctctcccctaaac
SEQ ID NO: 7: tggcatcctcttgttgctatc
SEQ ID NO: 8: cgaatcctttgagcatcttagtc
SEQ ID NO: 9: ggagattactgccctggctccta
SEQ ID NO: 10: gactcatcgtactcctgcttgctg

What is claimed is:

1. A method for mesotherapy in a subject in need thereof, comprising intracutaneous or subcutaneous administration a collagen-based composition for mesotherapy through intra cutaneous or subcutaneous injection to a site in need of increasing moisturization, preventing fine lines formation, enhancing skin brightening, providing anti-aging functions, treating skin disease, promoting lipolysis, nourishing hair follicle cell, promoting blood supply or preventing hair loss,
wherein the collagen-based composition for mesotherapy comprises
(i) 0.5 mg/mL˜4.8 mg/mL neutral pH collagen or modified collagen solution; and optionally
(ii) 2 mg/mL˜50 mg/ml biocompatible active ingredient(s) capable of increasing moisturization, preventing skin fine lines formation, enhancing skin brightening, providing anti-aging functions, treating skin disease, promoting lipolysis, nourishing hair follicle cell, promoting blood supply or preventing hair loss; and/or
(iii) local anesthesia drugs for reducing local pain and discomfort during and after treatment.

2. The method of claim 1, wherein the composition is administrated intracutaneously or subcutaneously through penetration into a transdermal channel opened by external forces.

3. The method of claim 2, wherein the external forces to open transdermal channels are selected from:
a) using single or array of nano- or micro-needles to puncture skin; and/or
b) photoelectric treatment including fractional CO₂ laser, picosecond laser, microcurrent treatment, electroporation and radio frequencies; and/or
c) dermabrasion, chemical peal.

4. The method of claim 1, wherein the composition is administrated intracutaneously or subcutaneously through injection by the ways selected from:
a) traditional single-needle or multi-needle by hands; and/or
b) auto or semi-auto intradermal injection system; and/or
c) needle-less intradermal injection system.

5. The method of claim 4, wherein the composition is administrated intracutaneously or subcutaneously through 31 gauge, 32 gauge, 33 gauge and/or 34 gauge needle injection; and/or
wherein the average extrusion force of the composition through a 32 gauge needle is ranged from about 2.0 N to about 20.0 N, from about 2.1 N to about 15.0 N, from about 2.2 N to about 14.0 N; and/or
wherein the lag time of the composition is ranged from about 20 minutes to about 90 minutes, from about 20 minutes to about 60 minutes, or from about 20 minutes to about 55 minutes.

6. The method of claim 1, wherein the source of collagen for part (i) is selected from allogenetic, mammal hides or marine species or axolotl hides derived matrix; and/or
the collagen is selected from full collagen or atelocollagen, or recombinant collagen or recombinant collagen peptides from microorganism, plants, insect cells or animal cells, or recombinant collagen peptides, or collagen mimic peptides.

7. The method of claim 1, wherein the modified collagen is derivatized with acetylation agent(s) that alter the pKa of collagen, and has one or more of the following features:
(a) soluble at neutral pH;
(b) does not undergo fibrillogenesis at physiological pH; and/or
(c) precipitates at acidic pH.

8. The method of claim 1, wherein the modified collagen is derivatized with one or more agents selected from the group consisting of glutaric anhydride, succinic anhydride, maleic anhydride, citric acid anhydride, oxalic acid anhydride and ethylenediamine tetraacetic anhydride.

9. The method of claim 1, wherein the neutral pH collagen solution comprises a neutralized solution comprising an acid soluble collagen, EDTA, sodium citrate, and wherein the acid soluble collagen comprises collagen selected from the group consisting of Type I collagen, Type III collagen and combinations thereof.

10. The method of claim 9, wherein said EDTA is disodium EDTA; and/or
wherein said EDTA is in a concentration between 15 and 50 mM; and/or
wherein said sodium citrate is in a concentration between 50 mM and 150 mM; and/or
wherein said neutral pH collagen solution further comprises a disaccharide, fructose, or combinations thereof; and/or
wherein said rapidly polymerizing collagen gel has an osmolality of 200-400 mOsmol/kg.

11. The method of claim 9, wherein the neutral pH collagen solution is a re-dissolved freeze-dried acid soluble collagen solution and neutralized with sodium bicarbonate injection or sodium citrate injection.

12. The method of claim 1, wherein the biocompatible active ingredient(s) is one or more selected from the group consisting of:
the biocompatible active ingredient(s) capable of increasing moisturization selected from the group consisting of: polyols, natural or derivatized/modified hyaluronic acid, heparosan, glycerin, derivatized chitosan, chondroitin sulfate, dermatan sulfate, nicotinamide;
the biocompatible active ingredient(s) capable of preventing or neutralizing fine lines formation selected from the group consisting of: botulinum toxin or recombinant botulinum toxin, hexapeptide-3 (Argireline), retinol, ilomastat (ilomostat), 1,2-diacyl-sn-glycero-3-phosphocholine, carnosine, carnitine, glycine, proline, valine;
the biocompatible active ingredient(s) capable of improving skin laxity and subcutaneous volume loss selected from the group consisting of: elastin or tropoelastin, crosslinked hyaluronic acid, crosslinked chitosan, hydroxyapatite microspheres, PLLA microspheres, PLGA spheres, PLA-PEG spheres, PCL spheres, PDO spheres;
the biocompatible active ingredient(s) capable of promoting anti-aging and rejuvenation function selected from the group consisting of: poly deoxyribonucleic acid (PDRN), polynucleotide (PN), dimethylethanolamine, growth factors, copper peptides;
the biocompatible active ingredient(s) capable of helping anti-oxidation and skin whitening selected from the group consisting of: azelaic acid, Vitamin C, glycolic acid, alpha-arbutin, kojic acid, glutathione, lipoic acid, N-acetylcysteine, superoxidase dismutase, methylsulfonylmethane, tretinoin or retinal, dimethicone, nicotinamide, tetrahydrocurminoids;
the biocompatible active ingredient(s) capable of supplying skin and follicle nutrition and enhancing regeneration selected from the group consisting of: decorin, biotin, amino acids, panthenol, caffeine, vitamins (B vitamins, Vitamin C, Vitamin E, Vitamin K), taurine, mineral (calcium chloride, copper sulfate, ferrous sulfate, magnesium sulfate, nickel chloride, sodium metasilicate, zinc sulfate), adenine, linoleic acid, pyruvate, platelet-rich plasma, cell-free fat extract, stem cell-derived exosomes, buflomedil; and
the biocompatible active ingredient(s) capable of treating and remitting cellulite selected from the group consisting of: collagenase, L-carnitine, phosphatidylcholine, sodium deoxycholate, hyaluronidase for cellulite treatment.

13. The method of claim 12, wherein the polyols are sugar alcohols selected from glycerin, fructose, xylitol sorbitol, mannitol, allitol, and palatinitol; and/or wherein said polyol is in a concentration between 1.5% and 4% (w/v).

14. The method of claim 12, wherein the molecular weight of hyaluronic acid, heparosan and/or chitosan is range from 5 kDa to 100 kDa.

15. The method of claim 12, wherein the size of the hydroxyapatite microspheres, PLLA microspheres, PLGA spheres, PLA-PEG spheres, PCL spheres, PDO spheres is ranged from 5 to 150 μm; and/or
wherein the non-resorbable or slowly resorbable spheres are obtained through spray-precipitation technique, emulsion, double emulsion evaporation method, microfluidic reaction, solid-gel process, melt extrusion technique, sintering process or stamp formation; and/or
wherein the non-resorbable or slowly resorbable spheres are sterilized through heat moist sterilization, gamma irradiation or ethylene oxide sterilization.

16. The method of claim 1, further comprising local anesthesia drugs such as lidocaine, procaine, preferably in a concentration of from 0.1% to 2% by weight.

17. A collagen-based composition for mesotherapy through intra cutaneous or subcutaneous injection, comprising
(i) 0.5 mg/mL˜4.8 mg/mL neutral pH collagen or modified collagen solution; and optionally
(ii) 2 mg/mL˜50 mg/ml biocompatible active ingredient(s) capable of increasing moisturization, preventing skin fine lines formation, enhancing skin brightening, providing anti-aging functions, treating skin disease, promoting lipolysis, nourishing hair follicle cell, promoting blood supply or preventing hair loss; and/or
(iii) local anesthesia drugs for reducing local pain and discomfort during and after treatment.

18. A method for the preparation of the composition used in the method of claim 1, comprising: combining part (i) with part (ii)/(iii), for example by
adding part (ii)/(iii) to part (i) by aseptic mixing;
adding part (iii) to part (i) and part (ii) by using part (iii) injection solution to dissolve the freeze-dried foam of combination of part (i) and part (ii);
adding part (iii) to part (i) and part (ii) by using part (iii) injection solution to dissolve the freeze-dried foam of combination of part (i), using alkali injection solution to dissolve freeze-dried combination of part (ii), mixing two solving liquid together before treatment.


SEQUENCE LISTING (10 sequences)

SEQ ID NO: 1 - DNA, 21 bp, synthetic construct
  source 1..21: mol_type = other DNA
  misc_feature 1..21: note = FW primer for Col alpha1 I (NM_053304.1)
atgtctggtttggagagagca

SEQ ID NO: 2 - DNA, 20 bp, synthetic construct
  source 1..20: mol_type = other DNA
  misc_feature 1..20: note = RV primer for Col alpha1 I (NM_053304.1)
gaggagcaggacttcttgag

SEQ ID NO: 3 - DNA, 22 bp, synthetic construct
  source 1..22: mol_type = other DNA
  misc_feature 1..22: note = FW primer for Col alpha1 III (NM_032085.1)
gcctcccagaacattacatacc

SEQ ID NO: 4 - DNA, 22 bp, synthetic construct
  source 1..22: mol_type = other DNA
  misc_feature 1..22: note = RV primer for Col alpha1 III (NM_032085.1)
gcctcccagaacattacatacc

SEQ ID NO: 5 - DNA, 23 bp, synthetic construct
  source 1..23: mol_type = other DNA
  misc_feature 1..23: note = FW primer for MMP-1alpha (NM_001134430.1)
ctcccttggactcactcattcta

SEQ ID NO: 6 - DNA, 23 bp, synthetic construct
  source 1..23: mol_type = other DNA
  misc_feature 1..23: note = RV primer for MMP-1alpha (NM_001134430.1)
agaacatcacctctcccctaaac

SEQ ID NO: 7 - DNA, 21 bp, synthetic construct
  source 1..21: mol_type = other DNA
  misc_feature 1..21: note = FW primer for TIMP-1 (NM_053819.1)
tggcatcctcttgttgctatc

SEQ ID NO: 8 - DNA, 23 bp, synthetic construct
  source 1..23: mol_type = other DNA
  misc_feature 1..23: note = RV primer for TIMP-1 (NM_053819.1)
cgaatcctttgagcatcttagtc

SEQ ID NO: 9 - DNA, 23 bp, synthetic construct
  source 1..23: mol_type = other DNA
  misc_feature 1..23: note = FW primer for Beta-Action (NM_031144.3)
ggagattactgccctggctccta

SEQ ID NO: 10 - DNA, 24 bp, synthetic construct
  source 1..24: mol_type = other DNA
  misc_feature 1..24: note = RV primer for Beta-Action (NM_031144.3)
gactcatcgtactcctgcttgctg